(19) United States
(12) Patent Application Publication
(10) Pub. No.: US 2011/0192413 A1
(43) Pub. Date: Aug. 11, 2011

(54) Two-Phase Composition for Improving Curl Retention

(75) Inventors: Ina Bräutigam, Darmstadt (DE); Sabine Förster, Pfungstadt (DE)

(73) Assignee: KPSS-KAO PROFESSIONAL SALON SERVICES GMBH, Darmstadt (DE)

(21) Appl. No.: 13/088,516

(22) Filed: Apr. 18, 2011

Related U.S. Application Data
(62) Division of application No. 11/872,277, filed on Oct. 15, 2007.

(30) Foreign Application Priority Data
Oct. 10, 2006 (EP) 06 021 171.1-1521

(51) Int. Cl. A61K 8/81; A61Q 5/06; A61Q 5/12; A45D 7/04

(52) U.S. Cl. 132/203; 424/70.15; 424/70.11; 424/70.16

(57) ABSTRACT

A two phase composition conditions and improves curl retention of permanently shaped and/or natural curly hair. The composition has 5 to 50%, by weight calculated to total composition, oil phase and 50 to 95%, by weight calculated to total composition, an aqueous phase wherein oil phase has at least one volatile oil at a concentration of 70 to 100%, by weight, calculated to the content of oil phase, and aqueous phase has 0.1 to 20% by weight calculated to the content of aqueous phase at least one fixing polymer, 10 to 40% by weight calculate to the content of aqueous phase at least one water miscible organic solvent and 0.05 to 2% by weight calculated to the content of the aqueous phase at least one cationic surfactant.

Present invention is related to a composition for conditioning and improving curl retention of curly hair comprising two optically separated phases when standing at zero shear rate which becomes homogeneous upon shaking.

Conditioning compositions have been known for many years. Among them it is possible to find products on hair care market for conditioning and especially improving curl retention of curly hair. On the other hand, there are products on hair care market for conditioning and styling hair.

For people having curly hair it is important that the curls do appear nice for along time. With nice curly hair appearance it is meant that curls are neatly distributed over the hair length. Within the meaning of the present description, with the term curly hair it is meant that the curly hair can be natural or permanently shaped or curled with a hot iron or with curling rod.

WO 02/060397 discloses dual phase conditioning and styling composition for heat styling hair. According to the document compositions comprise an oil phase comprising volatile oil and an aqueous alcoholic phase comprising styling polymer, an emulsifier and salt. The document does not address at all treatment of curly hair, let alone curl retention of curly hair.

EP 996 408 discloses dual phased compositions for make up cleansing wherein a polyvinylpyrrolidone copolymer is used as a demixing agent. The documents does not address at all hair care application of such composition.

The inventors of the present invention have surprisingly found out that a two phase composition comprising an oil phase and a water phase wherein two phases are optically separated at zero shear rate and becomes homogeneous upon shaking and returns again to optically separated two phases upon release of agitation improves curl retention of curly hair.

Accordingly, the object of the present invention is a composition for hair comprising 5 to 50%, by weight calculated to total composition, oil phase and 50 to 95%, by weight calculated to total composition, of aqueous phase wherein oil phase comprises at least one volatile oil at a concentration of 70 to 100%, by weight, calculated to the content of oil phase, and aqueous phase comprises 0.1 to 20% by weight calculated to the content of aqueous phase at least one fixing polymer, 10 to 40% by weight calculate to the content of aqueous phase at least one water miscible organic solvent and 0.05 to 2% by weight calculated to the content of the aqueous phase at least one cationic surfactant wherein oil and aqueous phases are optically separated at zero shear rate and becomes homogeneous upon shaking and returns again to optically separated two phases upon release of agitation.

Further objective of the present invention is the use of the composition comprising 5 to 50%, by weight calculated to total composition, oil phase and 50 to 95%, by weight calculated to total composition, of aqueous phase wherein oil phase comprises at least one volatile oil at a concentration of 70 to 100%, by weight, calculated to the content of oil phase, and aqueous phase comprises 0.1 to 10% by weight calculated to the content of aqueous phase at least one fixing polymer, 10 to 40% by weight calculate to the content of aqueous phase at least one water miscible organic solvent and 0.05 to 2% by weight calculated to the content of the aqueous phase at least one cationic surfactant wherein oil and aqueous phases are optically separated at zero shear rate and becomes homogeneous upon shaking and returns again to optically separated two phases upon release of agitation for improving curl retention of curly hair.

Compositions of the present invention comprises optically separated two phases which becomes homogeneous upon shaking and returns again to optically separated two phases upon release of agitation. Compositions of the present invention returns to optically separated two phases upon release of agitation preferably within 24 hours, more preferably within 20 hours and most preferably within 10 hours. It should be noted that with the compositions of the present invention separated oil and aqueous phases are recognizable after 15 to 30 minutes of shaking. The above referred separation periods refer to the complete separation of the two phases i.e. returning to their original state.

Compositions of the present invention comprises oil phase at a concentration of 5 to 50%, preferably 10 to 40% more preferably 15 to 30% by weight calculated to total composition. Oil phase comprises 70 to 100%, preferably 80 to 100% and more preferably 90 to 100% by weight calculated to the content of oil phase at least one volatile oil. Suitable volatile oils are silicones such as dimethicone and cyclomethicone and mixtures thereof, and volatile hydrocarbons such as isododecane, isohexadecane and isoprafin. The most preferred are silicones and from those dimethicone and cylomethicone and mixtures thereof.

Suitable volatile dimethicones are the ones with a viscosity of less than 5 mm²/s. The most preferred are dimethicone with a viscosity of 1 mm²/s and 0.65 mm²/s which are available from Dow Corning with the trade name DC 200 Fluid.

Suitable volatile cyclomethicones are according to general formula

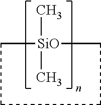

where n is a number between 3 and 7. Preferred are cyclopentasiloxanes known with the trade name for example Dow Corning 245.

Non-volatile silicones may also be incorporated into the compositions of the present invention at a low concentration, i.e. lower than 1% by weight calculated to the content of oil phase. Suitable ones are arylated silicones such as phenyltrimethicone known with the trade name Dow Corning 556, and dimethicones with higher viscosity.

In addition, natural oils such as olive oil, almond oil, avocado oil, wheatgerm oil, ricinus oil can be incorporated into the oil phase. Concentration of natural oils are below 1%, preferably below 0.5% by weight calculated to the content of the oil phase. Solubility of the natural oil in used volatile oil should be examined in selecting natural oil. As a rule selected natural oil should be soluble in the volatile oil used in the composition.

Compositions of the present invention comprise aqueous phase at a concentration of 50 to 95%, preferably 60 to 90% more preferably 70 to 85% by weight calculated to total composition. Aqueous phase comprises 0.1 to 20%, preferably 0.5 to 15%, more preferably 0.5 to 10% by weight calculated to the content of aqueous phase at least one fixing polymer. The composition can also comprise mixture of more than one fixing polymer. In selecting fixing polymers attention should be paid to the solubility of the polymer in aqueous phase as described above and also in the total composition wherein oil and aqueous phases are present together and mixed to become homogeneous composition by mixing.

Suitable polymers are non-ionic, cationic and amphoteric polymers. Anionic polymers may also be used so far if no incompatibility is observed in the presence of cationic surfactant. One of the important point in selecting fixing polymer is that the polymer should not thicken the aqueous phase. In other words the viscosity of the aqueous phase should remain low i.e. below 100 mPa·s, preferably 50 mPa·s.

Suitable non-ionic polymers are polyvinylpyrrolidone, polyvinylpyrrolidone/vinylacetate copolymer and polyvinylpyrrolidone/vinylacetate/vinylpropionate copolymer. Preferred is polyvinylpyrrolidone/vinylacetate copolymer. In addition to these fixing polymers derivatives of natural polymers such as hydroxylethylcellulose may also be used in combination with one of the fixing polymers mentioned above.

Suitable cationic polymers include Polyquaternium-4, Polyquaternium-11, Polyquaternium-16, Polyquaternium-18, Polyquaternium-24, Polyquaternium-28, Polyquaternium-46, polyvinylpyrrolidone/dimethylaminoethyl methacrylate, and cationically derivatized natural polymers. Preferred are Polyquaternium-4, Polyquaternium-11 and Polyquaternium-16.

Suitable amphoteric polymers are those available from the company National Starch under the trade name Amphomer such as Octylacrylamide/Acrylates/Butylaminoethyl methacrylate copolymer, those available under the trade name Diaformer (methacryloylethylbetaine/methacrylates copolymer). Preferred are the ones available under the trade name Amphomer.

In another preferred from of the invention, aqueous phase of the composition comprises combination of non-ionic and cationic fixing polymers.

As a fixing polymer silicone containing polymers either grafted or block copolymer are also useful. Suitable and the preferred one is Polysilicone-9.

Aqueous phase of the compositions of the present invention comprises 10 to 40%, preferably 15 to 35%, more preferably 20 to 30% by weight calculate to the content of aqueous phase at least one water miscible organic solvent-. With the term “water miscible” it is meant that the organic solvent is miscible with water at any ratio. Suitable ones are C₂-C₄ monohydric alcohols such as ethanol, propanol, isopropanol, butanol and isobutanol, benzyl alcohol and benzyloxyethanol and their mixtures. Most preferred are ethanol and isopropanol.

Aqueous phase of the compositions of the present invention comprises at least one cationic surfactant at a concentration of 0.05 to 2%, preferably 0.1 to 1%, more preferably 0.2 to 0.75% by weight calculated to the content of the aqueous phase. Suitable cationic surfactants are according to the general formula

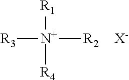

where R₁ is a saturated or unsaturated, branched or non-branched alkyl chain with 8-22 C atoms or

R₅CONH(CH₂)ₙ

where R₅ is saturated or unsaturated, branched or non-branched alkyl chain with 7-21 C atoms and n has typical value of 0-4 or

R₆COO(CH₂)ₙ

where R₆ is saturated or unsaturated, branched or non-branched alkyl chain with 7-21 C atoms and n has typical value of 0-4, and
R₂ is a hydrogen, saturated or unsaturated, branched or non-branched alkyl chain with 1-22 C atoms or

R₅CONH(CH₂)ₙ

where R₅ is saturated or unsaturated, branched or non-branched alkyl chain with 7-21 C atoms and n has typical value of 0-4 or

R₆COO(CH₂)ₙ

where R₆ is saturated or unsaturated, branched or non-branched alkyl chain with 7-21 C atoms and n has typical value of 0-4,
and R₃ and R₄ are independent from each other H or lower alkyl chain with 1 to 4 carbon atoms, and X is chloride, bromide or methosulfate.

Suitable cationic surfactants are particularly cetyl trimethly ammonium chloride, steartrimonium chloride, behentrimoinium chloride, stearamidopropyl trimonuim chloride, behenamidopropylethyldimonium ethosulfate, behenamidopropyltrimonium methosulfate, cocamidopropyltrimonium chloride, cocotrimonim chloride, palmitamidopropyltrimonum chloride, dipalmitoyltrimonium chloride, di-C12-C15 alkyldimoniumchloride, distearyldimonium chloride, dipalmitoylethylhydroxyethylmonium chloride, dioleoylethyl dimethyl ammonium methosulfate, dioleoylethyl hydroxyethylmonium methosulfate, dilinolamidopropyldimonium chloride, dioleylethyl hydroxyethylmonium chloride and dipalmitoylethyldimonium chloride.

In another preferred from of the invention the aqueous phase is comprised mixture of two cationic surfactants such as steartrimonium chloride and di-C12-C15 alkyldimoniumchloride.

The pH of the aqueous phase varies from 3 to 7, particularly 4.5 to 6. For adjusting the pH of the said conditioner compositions, following ingredients can be used: Organic acids such as citric acid, lactic acid, tartaric acid, malic acid, maleic acid, fumaric acid, levulinic acid, butyric acid and hydroxy butyric acids, valeric acid, oxalic acid, succinic acid, mandelic acid, glycolic acid, glucuronic acid, propionic acid, pyruvic acid, salicylic acid or acetic acid or inorganic acids such as hydrochloric acid, phosphoric acid, sulphuric acid, nitric acid. The pH of the conditioner composition can also be adjusted to the required pH by using alkaline solution such as sodium hydroxide, potassium hydroxide, ammonium hydroxide or their salts with those acids mentioned above.

Compositions of the present invention can comprise UV filters for protection of hair from environmental influences. It should be noted that oil soluble UV filters are contained in the oil phase and water soluble ones are added to the aqueous phase although partitioning the UV filter between two phases is not excluded. The UV-absorbing substance is preferably selected from the following compounds: 4-Aminobenzoic acid and the esters and salts thereof, 2-phenyl benzimidazole-5-sulfonic acid and the alkali and amine salts thereof, 4-dimethyl aminobenzoic acid and the esters and salts thereof, cinnamic acid and the esters and salts thereof, 4-methoxycinnamic acid and the esters and salts thereof, salicylic acid and the esters and salts thereof, 2,4-dihydroxybenzophenone, 2,2′,4,4′-tetrahydroxy-benzophenone, 2-hydroxy-4-methoxybenzophenone and its 5-sulfonic acid or the sodium salt thereof, 2,2′-dihydroxy-4,4′-dimethoxybenzophenone, 2-hydroxy-5-chlorobenzophenone, 2,2′-dihydroxy-4-methoxybenzophenone, 2,2′-dihydroxy-4,4′-dimethoxy-5,5′-disulfobenzo-phenone or the sodium salt thereof, 2-hydroxy-4-octyloxybenzophenone, 2-hydroxy-4-methoxy-4′-methylbenzophenone, 3-benzyl-idenecampher, 3-(4′-sulfo)-benzyl-idenebornane-2-one and the salts thereof, 3-(4′-methyl benzylidene)-DL-campher and/or 2-ethylhexyl-2-cyano-3,3-diphenyl acrylate also known as Octocrylene.

The preferred amount of the UV-absorber ranges from about 0.01% to 2.5%, more preferably from 0.05% to 1% by weight, calculated to the total composition.

The moisturising agents may be included into the aqueous phase of the compositions. Suitable ones are panthenol, polyols, such as glycerol, polyethylene glycols with molecular weight 200 to 20,000. The moisturising ingredients can be included in the aqueous phase of the compositions at a concentration range of 0.01-2.5% by weight calculated to the content of the aqueous phase.

Natural plant extracts may as well form part of the compositions of the present invention. Natural extracts should be included into the aqueous phase of the compositions. Suitable aqueous (e.g. steam-distilled) alcoholic or hydro-alcoholic plant extracts known per se are in particular extracts from leaves, fruits, blossoms, roots, rinds or stems of almond, aloe, pineapple, artichoke, arnica, avocado, valerian, henbane, birch, stinging nettle, echinacea, ivy, wild angelica, gentian, ferns, pine needles, silver weed, ginseng, broom, bamboo, green tea, oat, rose hip, hamamelis, hay flowers, elderberry, hop, coltsfoot, currants, chamomile, carrots, chestnuts, clover, burr root, coconut, cocoanut, mango, peach, lemon, cornflower, wheat, apricot, lime blossom, lily of the valley, marine algae, balm, mistletoe, passion flower, ratanhia, marigold, rosemary, horse chestnut, pink hawthorn, sage, horsetail, yarrow, primrose, nettle, thyme, walnut, wine leaves, white hawthorn, etc.

The aqueous and oil phases of the compositions of the present invention may be coloured. The dyestuff suitable for product colouring purposes are all useful for this purpose. It should be noted that for colouring aqueous phase water soluble dyes should be used and for colouring oil phase oil soluble dyes are suitable. The nature of the dyestuff is actually not important but preferred are non substantive dyes, i.e. not remaining on hair after washing with water or shampooing.

Fragrance, chelating agent, preservatives and other conventional cosmetic ingredients can be included at their usual concentrations either into the oil or aqueous phases depending on their solubility.

The composition of the present invention is preferably transparent, i.e. both oil and aqueous phases are transparent as judged by a naked eye in a transparent glass or PET bottle with a thickness of up to 3 cm.

Composition of the present invention are leave-in composition, i.e. not rinsed off from hair after application. Accordingly, in a process compositions of the present invention are applied to the shampooed and/or wetted hair and hair is dried. It is possible to apply compositions of the present invention as a lotion and also from a bottle equipped with a pump spray. Preferred application is spraying onto hair.

Following examples are to illustrate the invention but not to limit,

### EXAMPLE 1

% by weight

Oil phase

| | |
|---|---|
| Trisiloxane | 99.8 |
| Fragrance | 0.2 |

Aqueous phase

| | |
|---|---|
| VP/VA copolymer | 3.5 |
| Steartrimoniumchloride | 0.2 |
| Di-C₁₂-C₁₅ alkyldimonium chloride | 0.1 |
| Ethanol | 23.0 |
| Citric acid/Ammonium hydroxide | q.s. to pH 6.0 |
| Water | to 100 |

The above compositions are transparent compositions. The above composition is used at a concentration of oil phase of 15% by weight and 85% aqueous phase.

Curl retention of a curly hair tress curled using a rod with a diameter of 1.1 cm was determined with the above composition as follows. The hair tress used was approximately 3 g and having a length of 20 cm before curling. The length of curled hair was 12 cm. To the towel dried hair approximately 0.5 g of the above composition was applied with by spraying after homogeneously mixing both phases by shaking and the tress was dried with a hair drier. Curl retention was determined under ambient conditions at a temperature of approximately 25° C. and at a relative humidity of approximately 60%. At predetermined time intervals the length of hair was measured and curl retention was calculated using the following equation.

Curl retention %=((L−Lₜ)/(L−L₀))×100

wherein L is the original length of hair streak, Lₜ is the length of curled hair at time t and L₀ is the length of curled at the beginning of the test.

For comparative purposes similar hair tress was used but not treated with the above composition.

Curl retention %

| Time (min) | Example 1 | Comparative |
|---|---|---|
| 0 | 100 | 100 |
| 5 | 98 | 97 |
| 10 | 96 | 90 |
| 15 | 95 | 81 |
| 20 | 92 | 76 |
| 25 | 90 | 71 |
| 30 | 89 | 68 |
| 35 | 87 | 64 |
| 40 | 84 | 63 |
| 45 | 83 | 61 |
| 50 | 81 | 58 |
| 55 | 79 | 54 |
| 60 | 77 | 53 |

From the above results it is clear that Example 1 improves curl retention.

### EXAMPLE 2

% by weight

Oil phase

| | |
|---|---|
| Trisiloxane | 99.8 |
| Fragrance | 0.2 |

Aqueous phase

| | |
|---|---|
| VP/VA copolymer | 3.5 |
| Polyqauternium-11 | 0.5 |
| Steartrimoniumchloride | 0.15 |
| Di-C12-C15 alkyldimonium chloride | 0.15 |
| Ethanol | 20.0 |
| Lactic acid/Ammonium hydroxide | q.s. to pH 6.0 |
| Water | to 100 |

### EXAMPLE 3

% by weight

Oil phase

| | |
|---|---|
| Dimethicone 1 cst | 99.8 |
| Fragrance | 0.2 |

Aqueous phase

| | |
|---|---|
| VP/VA copolymer | 3.0 |
| Polysilicone-9 | 0.5 |
| Cetrimonium chloride | 0.15 |
| Dioleylethyl hydroxyethylmonium chloride | 0.15 |
| Benzophenone-4 | 0.10 |
| Ethanol | 25.0 |
| Lactic acid/Ammonium hydroxide | q.s. to pH 6.0 |
| Dyestuff CI 19140 and CI 42053 | q.s. |
| Water | to 100 |

### EXAMPLE 4

% by weight

Oil phase

| | |
|---|---|
| Dimethicone 1 cst | 99.6 |
| Benzophenone-3 | 0.2 |
| Fragrance | 0.2 |

Aqueous phase

| | |
|---|---|
| VP/VA copolymer | 3.0 |
| Polysilicone-9 | 0.5 |
| Cetrimonium chloride | 0.15 |
| Dioleylethyl hydroxyethylmonium chloride | 0.15 |
| Benzophenone-4 | 0.10 |
| Ethanol | 25.0 |
| Lactic acid/Ammonium hydroxide | q.s. to pH 7.0 |
| Dyestuff CI 19140 and CI 42053 | q.s. |
| Water | to 100 |

1-9. (canceled)

10. A process for treating hair, the process comprising:
applying a two phase composition to shampooed or wetted hair, wherein the two phase composition comprises 15 to 50%, by weight calculated to the total composition, oil phase and 50 to 95%, by weight calculated to the total composition, an aqueous phase, wherein the oil phase comprises at least one volatile oil at a concentration of 90 to 100%, by weight, calculated to the content of the oil phase, and the aqueous phase comprises 0.1 to 20%, by weight calculated to the content of the aqueous phase, at least one fixing polymer, 10 to 40% by weight calculated to the content of the aqueous phase, at least one water miscible organic solvent and 0.05 to 2%, by weight calculated to the content of the aqueous phase, at least one cationic surfactant, wherein the oil and aqueous phases are optically separated at a zero shear rate, become homogeneous upon shaking and return again to optically separated two phases at a zero shear rate, and further wherein the at least one water miscible organic solvent is at least one monohydric alcohol.

11. The process according to claim 10, wherein the at least one cationic surfactant in aqueous phase is selected from compounds according to general formula 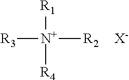
where R₁ is a saturated or unsaturated, branched or non-branched alkyl chain with 8-22 C atoms or
R₅CONH(CH₂)ₙ
where R₅ is saturated or unsaturated, branched or non-branched alkyl chain with 7-21 C atoms and n has typical value of 0-4 or
R₆C(O)O(CH₂)ₙ
where R₆ is saturated or unsaturated, branched or non-branched alkyl chain with 7-21 C atoms and n has typical value of 0-4, and
R₂ is a hydrogen, saturated or unsaturated, branched or non-branched alkyl chain with 1-22 C atoms or
R₅CONH(CH₂)ₙ
where R₅ is saturated or unsaturated, branched or non-branched alkyl chain with 7-21 C atoms and n has typical value of 0-4 or
R₆C(O)O(CH₂)ₙ
where R₆ is saturated or unsaturated, branched or non-branched alkyl chain with 7-21 C atoms and n has typical value of 0-4,
and R₃ and R₄ are independent from each other H or lower alkyl chain with 1 to 4 carbon atoms, and X is chloride, bromide or methosulfate.

12. The process according to claim 10, wherein the volatile oil in the oil phase is selected from silicone compounds.

13. The process according to claim 10, wherein the fixing polymer in the aqueous phase is selected from non-ionic, cationic or amphoteric polymers.

14. The process according to claim 10, further comprising:
at least one ultraviolet filter either in the oil phase or in the aqueous phase.

15. The process according to claim 10, wherein the aqueous phase has a pH between 3 and 7.

16. The process according to claim 10, wherein the composition is transparent.

17. The process according to claim 10, wherein the aqueous phase comprises greater than 20% and not more than 40%, by weight calculated to the content of the aqueous phase, at least one water miscible organic solvent.

18. The process according to claim 10, wherein the at least one monohydric alcohol is selected from a group consisting of ethanol, propanol, isopropanol, butanol, isobutanol, benzyl alcohol, benzyloxyethanol and mixtures thereof.

19. The process according to claim 10, further comprising:
towel drying the hair before the composition is applied to the hair.

20. The process according to claim 10, further comprising:
leaving the composition in the hair after the composition is applied to the hair.

21. The process according to claim 10, further comprising:
drying the hair without rinsing the composition off the hair.

22. The process according to claim 10, wherein the composition is applied to the hair as a lotion or sprayed onto the hair.

23. The process according to claim 10, further comprising:
homogeneously mixing the oil and aqueous phases of the composition before the composition is applied to the hair.

24. The process according to claim 10, wherein application of the two phase composition to the hair improves curl retention of the hair

25. A process for treating hair, the process comprising:
applying a two phase composition to shampooed or wetted hair, wherein two phase composition for hair comprises 15 to 50%, by weight calculated to the total composition, oil phase and 50 to 95%, by weight calculated to the total composition, an aqueous phase, wherein the oil phase comprises of at least one volatile oil at a concentration of 90 to 100%, by weight calculated to the content of the oil phase, and the aqueous phase comprises 0.1 to 20%, by weight calculated to the content of the aqueous phase, at least one fixing polymer selected from the group consisting of at least one non-ionic polymer, at least one cationic polymer, at least one amphoteric polymer, at least one silicone containing polymer and combinations thereof, 10 to 40%, by weight calculated to the content of the aqueous phase, at least one water miscible organic solvent and 0.05 to 2%, by weight calculated to the content of the aqueous phase, at least one cationic surfactant wherein the oil and aqueous phases are optically separated at a zero shear rate and become homogeneous upon shaking and return again to optically separated two phases at a zero shear rate,
wherein the at least one non-ionic polymer is selected from the group consisting of polyvinylpyrrolidone, polyvinylpyrrolidone/vinylacetate copolymer, polyvinylpyrrolidone/vinylacetate/vinylpropionate copolymer and combinations thereof,
wherein the at least one cationic polymer is selected from the group consisting of Polyquaternium-4, Polyquaternium-11, Polyquaternium-16, Polyquaternium-18, Polyquaternium-24, Polyquaternium-28, Polyquaternium-46, polyvinylpyrrolidone/dimethylaminoethyl methacrylate, cationically derivatized natural polymers and combinations thereof,
wherein the at least one amphoteric polymer is selected from the group consisting of octylacrylamide/acrylates/butylaminoethyl methacrylate copolymer, methacryloylethylbetaine/methacrylates copolymer and mixtures thereof, and
further wherein the at least one silicone containing polymer is selected from the group consisting of a silicone containing grafted copolymer, a silicone containing block copolymer, Polysilicone-9 and combinations thereof.

26. The process according to claim 25, wherein application of the two phase composition to the hair improves curl retention of the hair
